(19) United States
(12) Patent Application Publication
(10) Pub. No.: US 2005/0100571 A1
(43) Pub. Date: May 12, 2005

(54) Method and device to treat skin affected by a corn

(76) Inventor: Larry Keyes, Oak Park, IL (US)

Correspondence Address: BARNES & THORNBURG, P.O. BOX 2786, CHICAGO, IL, 60690-2786, US

(21) Appl. No.: 10/984,143

(22) Filed: Nov. 9, 2004

Related U.S. Application Data
(60) Provisional application No. 60/519,070, filed on Nov. 10, 2003.

(51) Int. Cl. A61K 31/202; A61K 7/00; A61K 35/78; A61K 33/32

(52) U.S. Cl. 424/401; 424/776; 424/642; 514/560

(57) ABSTRACT

A method of treating skin affected by a corn comprising applying a cosmetic preparation to the skin, and applying silicone or other occlusive material to the skin. The cosmetic preparation and occlusive material may be in the form of layers. The method may be in the form of a device or kit for treating the skin.

### CLAIM FOR PRIORITY

This application claims priority to provisional patent application Ser. No. 60/519,070 filed Nov. 10, 2003, which is incorporated herein by reference.

### BACKGROUND

A corn is a skin lesion that typically is found on feet and caused by chronic pressure and friction of the shoe or by adjacent toe joints. The corn may, for example, occur over a toe joint, at the end of the toe, or over a bony prominence on the side of the toe. Removal of corns may be desirable either because of their unattractiveness, the pain caused by the corns, or both. There may be different types of corns, depending upon the cause of the corn.

Regardless of the cause, the structure of the corn is similar in that it includes the following: an outer most layer of skin (i.e, the stratum corneum) which typically becomes thickened, hardened and discolored with multiple layers of keratinous tissue, the other epidermal and dermal layers which typically becomes hypertrophied, thickened and/or painful; and the skin around the corn that may become discolored and swollen.

The existing non-surgical chemically based corn treatments work by targeting the outer hard keratinized skin layers and chemically burning them away and/or causing a separation of the layer from the rest of the skin. This has the beneficial effect of reducing the bulk of the lesion and thus relieving the pain to an extent. However, these treatments do not directly treat the remaining structure of the corn (the deeper layers) and in fact can have a generally non-beneficial destructive effect on these layers if the chemical comes in contact with it. Local abscesses and pigmentary changes may occur. This is particularly true in the African American population whereas these chemicals can induce post traumatic hyperpigmentation. These existing chemical corn treatments typically are only useful for corns that have a thick outer layer (stratum corneum).

Because of the resulting hyperpigmentation, the existing chemical corn treatments are not effective from a cosmetic standpoint, especially the type of corn where the outer skin layer is thickened only slightly but the deeper layers have increased in thickness and have pigmentary color change, and/or demonstrate pain. With this latter type of corn, the hyperpigmentation is greater.

Accordingly, the present disclosure relates to the treatment of skin affected by corns. The present disclosure tends to be more effective in connection with corns that tend to have a greater pigmentary color change and thicker and deeper epidermal and dermal layers. Notwithstanding the foregoing, however, the present disclosure is not limited to any particular type of corn condition.

### SUMMARY

The present disclosure comprises one or more of the following features or combinations thereof disclosed herein or in the Detailed Description below.

The present disclosure in accordance with an embodiment relates to a method of treating skin affected by corn. The method comprises applying a cosmetic preparation to the skin affected by the corn; and applying silicone or other occlusive material to the skin. The cosmetic preparation and occlusive material may be in the form of layers. Additionally, in accordance with other embodiments, the present disclosure may be in the form of a device or kit for treating skin after the removal of the corn.

Additional features of the present disclosure will become apparent to those skilled in the art upon consideration of the following detailed description of illustrative embodiments of the disclosure.

### BRIEF DESCRIPTION OF THE DRAWINGS

FIG. 1 is a top view of an area of skin, a portion of which has been damaged during the removal of a corn;

FIG. 2 is a cross-section view of a covering in accordance with an embodiment of the present disclosure applied to the damaged skin of FIG. 1 comprising a silicone layer with a layer of the cosmetic preparation bonded with the silicone layer and tape for securing the silicone layer to the skin;

FIG. 3 is a cross section view of a covering in accordance with an other embodiment of the present disclosure applied to the damaged skin of FIG. 1 comprising a silicone layer and a separate layer of the cosmetic preparation; and

FIG. 4 is a cross section view of a covering in accordance with an other embodiment of the present disclosure including a silicone layer, a separate layer of the cosmetic preparation and a compression layer positioned over the silicone layer.

### DETAILED DESCRIPTION

While the concepts of the present disclosure are illustrated and described in detail in the drawings and the description below, such an illustration and description is to be considered as exemplary and not restrictive in character, it being understood that only the illustrative embodiment is shown and described and that all changes and modifications that come within the spirit of the disclosure are desired to be protected.

In the illustrated embodiment, a covering or other device to treat skin 10 affected by a corn comprises a tape 20, a silicone layer 22, and a layer of cosmetic preparation 24. The device may further comprise a compression layer 30. The device may be used to treat skin 10 affected by a corn (affected skin 26) to provide appropriate pigmentation to the skin. The affected skin 26 may include the stratum corneum and the other epidermal and dermal layers. In particular, the affected skin 26 may include the corn and skin adjacent or surrounding the corn. Alternatively, at least some of the corn may be removed prior to using the device and the affected skin 26 may include only the skin previously adjacent or surrounding the corn. Thus the device may serve both to remove the corn and to treat the skin previously adjacent or surrounding the skin, or, alternatively, to treat the skin previously adjacent or surrounding the corn after efforts have been made to remove the corn.

The device may be in the form of a kit or the like that includes the tape 20, the silicone layer 22, the layer of the cosmetic preparation 24, and the compression layer 30, each of which may have any suitable configuration and formulation. The device may also be in the form of a bandage or other covering that includes the silicone layer 22, the layer of the cosmetic preparation 24 in side-by-side contact with the silicone layer, the compression layer 30, and the tape 20 as an integrated patch or the like that can be applied over affected skin 26. The covering may include more or less than all of these components in accordance with other embodiments.

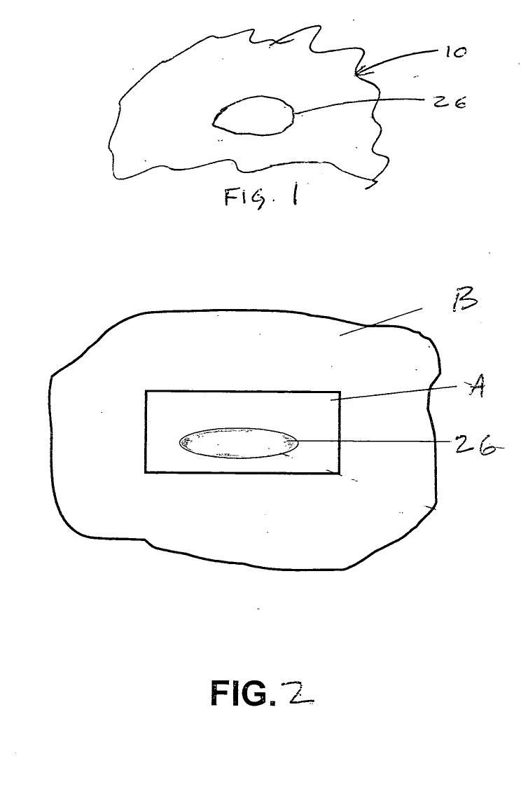

Either before the corn has been removed or after efforts have been made to remove the corn, the affected skin 26 may be treated as described herein. For example, the covering as described herein may be applied over the affected skin 26 that is surrounded by two areas of application. In this regard, a zone A may be a rectangular area that surrounds the affected skin 26. The zone A can be of any other suitable shape. The edges of zone A are a predetermined distance from the affected skin 26 such as, for example, 0.5-1.0 cm from any part of the affected skin 26. The silicone layer 22 and the layer of the cosmetic preparation 24 may cover the zone A. A zone B may be an area that surrounds zone A. The zone B encloses the zone A and has a predetermined inner border (see FIG. 2). The zone B extends in all directions for several millimeters or centimeters from the affected skin 26. The shape and size of the zone B depends on the nature of the affected skin 26, the anatomical area of the affected skin 26, and the cosmetic preparation 24. The covering may be applied to cover the zones A and B.

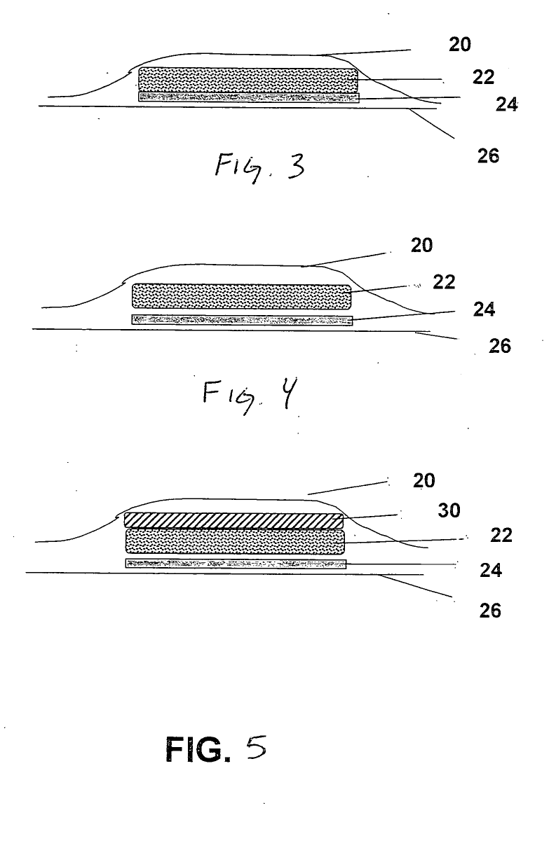

In the illustrated embodiment, the covering, comprising the tape 20, silicone layer 22, and the layer of the cosmetic preparation 24, is applied over the affected skin 26, such that the layer of the cosmetic preparation 24 is in contact with the affected skin 26 and enclosed by the silicone layer 22 (e.g., FIGS. 3 and 4). The compression layer 30 may provide compression (e.g., FIG. 5). The layer of the cosmetic preparation 24 may be bonded with the silicone layer 22 (e.g., FIG. 3) or may be applied separately (e.g., FIGS. 4 and 5). The compression layer 30 is positioned over the silicone layer 22 to provide compression (e.g., FIG. 5). The tape 20 is applied to hold the silicone layer 22, the layer of the cosmetic preparation 24, and the compression layer 30 over the affected skin 26 and also to provide compressive force.

In the illustrated embodiment, the tape 20 is applied to hold the silicone layer 22 and the layer of the cosmetic preparation 24 over the affected area. The tape 20 may have an adhesive side. The tape 20 may have any suitable configuration such as, for example, a hypo-allergenic breathable tape or a micropore paper tape or any other suitable tape. The tape 20 may have limited elasticity to provide compressive force when applied over the silicone layer 22 or the compression layer 30. The shape and size of the tape 20 may depend on the nature of the affected skin 26 and the anatomical area of the affected skin 26.

In the illustrated embodiment, the silicone layer 22 may be a silicone gel sheet and may have any suitable configuration. Any suitable silicone gel, silicone occlusive, sheeting silicone material, or any combination thereof may be used as the silicone layer 22. A number of silicone layers are available commercially, such as, for example, Dow Corning Silastic Sheeting (Dow Coming), Cica-Care (Smith & Nephew), Epi-Derm (Biodermis), Nagosil (Nagor), Nusil Technology or any other standard medical grade silicone gel sheeting. Any other suitable breathable occlusive layers such as transderm and duoderm can also be used. The thickness of the silicone layer 22 may vary depending upon the affected skin 26 and the anatomic location of the affected skin 26 and the structural integrity of the silicone gel sheeting used. The therapeutic side of the silicone layer 22 may be adhesive to provide a sealable contact with the skin 26 and the top side may be plasticized to allow for an adhesion of tape 20 and to provide durability. The silicone layer 22 can be obtained in precut shapes, sizes, thickness, and molds for specific anatomical places or can be obtained as a larger sheet to be customized as needed.

In the illustrated embodiment, the layer of the cosmetic preparation 24 is positioned over the affected skin 26 of the skin 10 and underneath the silicone layer 22. The cosmetic preparation 24 can be a moisturizing cosmetic or a skin nutrient such as, for example, eucerin, shea butter, GLA, linoleic acid, zinc oxide, or any other suitable cosmetic or a combination of cosmetics or compositions. Other commercially available moisturizing compositions or skin nutrient compositions can also be used. The layer of the cosmetic preparation 24 can be impregnated, infused or bonded with the silicone layer 22 or can be applied separately on top of the affected skin 26 over the skin 10 (see FIGS. 2A and 2B). The layer of the cosmetic preparation 24 can be applied in any suitable form and in any suitable amount over the affected skin 26.

In the illustrated embodiment, the tape 20 is applied over the silicone layer 22 to hold the silicone layer 22 and the layer of the cosmetic preparation 24 on top of the skin 12 (FIG. 2). The tape 20 may have an adhesive underside or an adhesive cosmetic can be applied separately to the underside of the tape 20 or on zones A and B over the skin 26.

If included, the compression layer 30 may be positioned over the silicone layer 22 (see FIG. 2C). The compression layer 30 may include a closed cell foam or a sponge or any other suitable material capable of providing uniform compression on the silicone layer 22 over the affected skin 26. The tape 20 may provide a compressive force when applied over the compression layer 30 or the silicone layer 22. The required compression may vary depending on the affected skin 26 and the anatomic location of the affected skin 26.

Accordingly, the present disclosure includes a method of treating skin affected by a corn, including applying cosmetic preparation to the affected skin, and applying silicone or other occlusive material to the affected skin. The method can be used both to remove the corn and treat the skin previously adjacent or surrounding the skin. Alternatively, the method can be used after efforts have been made to remove the corn to treat the skin previously adjacent or surrounding the corn.

The method can be carried out through any suitable steps, including, for example, the following steps:
1. The silicone layer 22 is cut according to the shape of zone A such that the edge of the silicone layer 22 has some distance from the affected skin 26. The affected skin 26 may be cleansed of necrotic and foreign debris to provide a flat and clean surface for the silicone layer 22. The affected skin 26 may be healing under primary intention.
2. A suitable layer of the cosmetic preparation 24 is applied over the affected skin 26 without covering all of zone A. The layer of the cosmetic preparation 24 can be applied separately or can be bonded with the silicone layer 22.
3. The therapeutic side of the silicone layer 22 is placed over the affected skin 26. Any wrinkles of the silicone layer 22 may be flattened and the edges may be pressed to the skin 26 to form a seal that traps the layer of the cosmetic preparation 24. The silicone layer 22 may also be infused with the layer of the cosmetic preparation 24.
4. An adherent can be applied to cover the zone B excluding zone A to serve as an adhesive base for the tape 20. The tape 20 may also have an adhesive underside.
5. An optional compression layer 30 of suitable dimensions may be placed over the silicone layer 22 to provide compression.
6. The tape 20 is applied over zones A and B to hold the silicone layer 22 and any compression layer 30 over the affected skin 26.

The covering may also be applied over the affected skin 26 in any other suitable way. The nature of the tape 20, the thickness, shape, and size of the silicone layer 22, the amount and nature of the layer of the cosmetic preparation 24, and the compression layer 30 can have any suitable configuration or formulation. The covering may also have the silicone layer 22, the layer of the cosmetic preparation 24 in side-by-side contact with the silicone layer, the compression layer 30, and the tape 20 as an integrated device that can be applied as a patch over the affected skin 26. The covering can also be provided as a kit comprising the tape 20, the silicone layer 22, the cosmetic 24, the compression layer 30, suitable cleansing solutions and appropriate coloring creams.

The method and device in accordance with an embodiment of the present disclosure, among other things, treats deep layers of the corn after the outer hard layers of skin have been removed. The method and device tends to have less undesired destructive side effects that result from prior art methods and also treats the other components of the corn. The benefits of the present disclosure in accordance with an embodiment may result from a combination of protection from the friction and pressure, controlled direct pressure, chemical action under occlusion, hydration, and pigmentation curing through chemical means.

There are a plurality of other benefits advantages that may be inferred from one or more embodiments of the present disclosure arising from the various features of the apparatus, systems, and methods described herein. It will be noted that other embodiments of each of the systems, method claims and kits of the present disclosure may not include all of the features described yet still benefit from at least some of the inferred advantages of such features. Those of ordinary skill in the art may readily devise their own implementations of an apparatus, system, and method that incorporate one or more of the features of the present disclosure and fall within the spirit and scope of the disclosure.

1. A method of treating skin affected by a corn, the method comprising:
(a) applying a cosmetic preparation to the skin affected by the corn, and
(b) applying an occlusive material to the skin.

2. The method of claim 1 wherein the occlusive material comprises silicone.

3. The method of claim 1 wherein the cosmetic preparation comprises a moisturizing composition.

4. The method of claim 1 wherein the cosmetic preparation comprises a skin nutrient.

5. The method of claim 1 wherein the cosmetic preparation comprises at least one of the following: eucerin, shea butter, GLA, linoleic acid, and zinc oxide.

6. The method of claim 6 wherein the occlusive material comprises silicone.

7. A method of treating skin affected by a corn comprising:
(a) applying a layer of cosmetic preparation to the skin, and
(b) applying a layer of occlusive material to the skin.

8. The method of claim 7 further including cutting the layer of occlusive material prior to application to correspond to a zone having an area greater than an area of skin affected by the corn

9. The method of claim 8 wherein during the application of the layer of cosmetic preparation to the area of skin the layer of cosmetic preparation is applied on an area less than the area of the zone.

10. The method of claim 7 further including adhering the layer of occlusive material to the person from whom the corn was removed.

11. The method of claim 7 further including applying compression to the layer of occlusive material.

12. The method of claim 11 wherein applying compression includes applying a compressive layer to the layer of occlusive material.

13. The method of claim 7 wherein the occlusive material comprises silicone.

14. The method of claim 7 wherein the cosmetic preparation comprises a moisturizing composition.

15. The method of claim 7 wherein the cosmetic preparation comprises a skin nutrient.

16. The method of claim 7 wherein the cosmetic preparation comprises at least one of the following: eucerin, shea butter, GLA, linoleic acid, and zinc oxide.

17. The method of claim 16 wherein the occlusive material comprises silicone.

18. A method of treating skin affected by a corn comprising:
(a) providing a device comprising an occlusive layer and a layer of cosmetic preparation, and
(b) applying the device over the skin.

19. The method of claim 18 wherein the occlusive layer comprises silicone.

20. The method of claim 18 wherein the cosmetic preparation comprises a moisturizing composition

21. The method of claim 18 wherein the cosmetic preparation comprises a skin nutrient.

22. The method of claim 18 wherein the cosmetic preparation comprises at least one of the following: eucerin, shea butter, GLA, linoleic acid, and zinc oxide.

23. The method of claim 22 wherein the occlusive layer comprises silicone.

24. A device for treating skin affected by a corn, the device comprising:
(a) an occlusive layer,
(b) a layer of cosmetic preparation in side-by-side contact with the occlusive layer, and
(c) an adhesive for adhering the occlusive layer to a person from whom the corn was removed.

25. The device of claim 24 wherein the occlusive layer comprises silicone.

26. The device of claim 24 wherein the cosmetic preparation comprises a moisturizing composition

27. The device of claim 24 wherein the cosmetic preparation comprises a skin nutrient.

28. A kit for treating skin affected by a corn, the kit comprising:
(a) a layer of cosmetic preparation for application to the skin;
(b) an occlusive layer for application to the skin over the layer of the cosmetic preparation, and
(c) a tape for adhering the occlusive layer to a person from whom the corn was removed.

29. The kit of claim 28 further including a compressive layer for applying a compressive force to the skin over the occlusive layer.

30. The kit of claim 29 wherein the compressive layer comprises at least one of a foam or a sponge.
